(19) United States
(12) Patent Application Publication
(10) Pub. No.: US 2011/0306556 A1
(43) Pub. Date: Dec. 15, 2011

(54) NEUROPROTECTION FROM BRAIN ANOXIA AND REPERFUSION INJURY DURING STROKE AND COMPOSITIONS OF PKG PATHWAY ACTIVATORS AND METHOD OF USE THEREOF

(75) Inventors: Kenneth DAWSON-SCULLY, Boca Raton, FL (US); Sarah Louise MILTON, Coral Gables, FL (US)

(73) Assignee: Florida Atlantic University, Boca Raton, FL (US)

(21) Appl. No.: 13/157,073

(22) Filed: Jun. 9, 2011

Related U.S. Application Data
(60) Provisional application No. 61/353,033, filed on Jun. 9, 2010.

(51) Int. Cl. A61K 38/17; A61P 9/10; A61P 25/00; A61K 31/519; A61K 31/708

(52) U.S. Cl. 514/17.7; 514/252.16; 514/21.2; 514/48

(57) ABSTRACT

A pharmaceutical composition for treating or preventing one or both of neural anoxia and reperfusion injury, which includes a pharmacological activator of the PKG pathway, and methods of treating or preventing medical conditions using a pharmacological activator of the PKG pathway.

This application claims the benefit of Provisional application Ser. No. 61/353,033, entitled “NEUROPROTECTION FROM BRAIN ANOXIA AND REPERFUSION INJURY DURING STROKE AND COMPOSITIONS OF PKG PATHWAY ACTIVATORS AND METHOD OF USE THEREOF”, filed Jun. 9, 2010.

### FIELD OF THE INVENTION

In one of its aspects, the present invention relates to a composition for neuroprotection from stroke reperfusion injury during clot removal. In another of its aspects, the present invention relates to a method of preventing brain cell death during anoxia using a neuroprotection composition as described herein. In yet another of its aspects, the present invention relates to a method of providing neuroprotection from anoxia.

### BACKGROUND OF THE INVENTION

The pharmacological inhibition of phosphodiesterase (PDE) leads to the protection of brain cells during hypoxic episodes (Caretti et al., 2008). Exposure to hypoxia triggers a variety of adverse effects in the brain that arise from metabolic stress and induce neuron apoptosis. Researchers examined the protective effect of modulating the nitric oxide (NO)/cGMP pathway by sildenafil, a selective inhibitor of phosphodiesterase-5 (PDE-5), Sildenafil (Viagra) was administered to mice, 1.4-mg/kg intraperitoneal injections daily for 8 days. They found that upregulating the NO/cGMP pathway by PDE-5 inhibition during hypoxia reduces neuron apoptosis, regardless of HIF-1alpha, through an interaction involving ERK1/2 and p38.

The PKG enzyme pathway is known, and an outline of the PKG pathway as it is involved in ion channel regulation is provided to assist in understanding:

Briefly, nitric oxide (NO) is produced by various NO synthases (NOS), some of which are activated by a rise in intracellular Ca2+. Many NO effects are mediated through direct activation of the soluble guanylyl cyclase (sGC), an enzyme generating the second messenger cyclic guanosine-3″,5′-mono-phosphate (cGMP). sGC is stimulated by NO to catalyze the formation of cGMP. cGMP is a cyclic nucleotide second messenger with effects on many pathways, one of which is the cGMP-dependent protein kinase (PKG) enzyme pathway. PKG is an enzyme that transfers a phosphate group from ATP to an intracellular protein, increasing or decreasing its activity.

Both the DNA sequence and protein function of PKG are conserved across animal kingdom including mammalians. PKG genes have been isolated from various animals spanning a wide variety of taxa ranging from humans (Sandberg et al., 1989) to even the malaria-causing protozoans Plasmodium falciparum (Gurnett et al., 2002). The protein phylogenetic analysis using 32 PKG sequences that include 19 species has shown the highly conserved link between PKG and behaviour in fruit flies, honey bees and nematodes (Fitzpatrick et al., 2004).

It is known that PKG may modulate neural ion channel activity i.e. neural potassium (K+) activity (Renger, 1999). A variety of mechanisms for this effect have been suggested, ranging from direct phosphorylation of the K+ channel by PKG, to the opposing indirect dephosphorylation of the K+ and other channels by phosphatases such as PP2A, which are themselves activated by PKG phosphorylation (Schiffmann et al., 1998; White et al., 1993; White, 1999; Zhou et al., 1996; Zhou et al., 1998). It has been suggested on theoretical grounds that future work in the area of regulation of the PKG pathway might yield some neuroprotective effects but no supporting evidence was provided (Jayakar and Dikshit, 2004).

Effects of cGMP activators on neural ion channels were proposed to lead to pain relief compounds (Liu et al., 2004).

Most research on ion channel direct phosphorylation focuses on the enzyme PKA, which is known to modulate many ion channels (Wang et al., 1999; Zeng et al., 2004). PKA often functions in opposition to PKG, but may function in the same direction.

There is extensive literature related to PKG's known roles in the regulation of smooth muscle and other non-neural tissues, where it may modulate ion channels. A significant amount of medical and pharmaceutical publications relate to such drugs as sildenafil (Viagra) and nitroglycerine for treatment of penile dysfunction or angina (Corbin and Francis, 1999; Patel and Diamond, 1997).

U.S. Pat. No. 6,300,327 to Knusel et al. teaches compositions and methods for use in modulating neurotrophin activity. Neurotrophin activity is modulated by administration of an effective amount of at least one compound which potentiates neurotrophin activity. This patent specifically teaches the potentiation of NT-3 by KT5823, and suggests that this potentiation provides a model for therapeutic intervention in a variety of neuropathological conditions.

U.S. Pat. No. 6,451,837 to Baskys teaches inhibition of the mitogen-activated protein kinase (MAPK) cascade that can lead to nerve cell death. In this context, Okadaic acid, an inhibitor of protein phosphatase was found to increase nerve cell death.

U.S. Pat. No. 6,476,007 to Tao et al. investigated the role of the NO/cGMP signalling pathway in spinal cord pain.

It is known that K+ channel activity may be involved in neural protection. As canvassed further below, existing literature has considered what happens to neural function during heat-induced malfunction; mechanisms known to provide heat tolerance, much as induction of the heat shock genes and proteins; and investigations into a direct mechanism for neural thermoprotection by K+ channel modulation.

It is know that anoxia causes increased K+ efflux from cells in the nervous system which results in blocking neural output (Rodgers et al., 2009; 2010).

It is known that the upregulation of heat shock proteins in either neurons or glia (Dawson-Scully et al., in press) can extend the time until behavioural and synaptic failure during anoxia.

Abnormal K+ concentrations have been associated with various neural failure scenarios such as spreading depression, ischemia, diabetic coma, and hyperthermia (Somjen, 2001; Somjen, 2002).

Recent research by one of the inventors suggests that prior stress down-regulates neuronal K+ currents leading in thermal protection (Robertson, 2004). This was based on findings that prior heat shock provides protective effects against future heat shock and that neuronal K+ currents are effected by prior heat stress (Ramirez et al., 1999).

Other investigations have suggested that K+ channels are involved in neural excitability in response to other stresses such as reactive oxygen (Wang et al., 2000).

An abrupt rise in extracellular potassium ([K⁺]ₒ) and depression of electrical activity in nervous tissue, shares many characteristics of cortical spreading depression (CSD³).

In mammalian tissue CSD has been associated with several important pathologies including stroke, seizures and migraine.

PKG inhibition results in reductions of seizure-like events from hyperthermia in locust neural motor patterns (Dawson-Scully et al., 2007).

Stroke is a human pathology that results in oxygen deprivation to brain cells (Baron and Moseley, 2000)

During the acute phase shortly after the onset of an ischemic stroke, tissue in the penumbra surrounding an infarct receives sufficient blood flow to survive, but not enough to function. As time passes, neurons in this penumbra die (Baron and Moseley, 2000).

Increasing evidence from investigations in human subjects suggests that typical migraine auras may be the clinical manifestation of a cortical spreading depression (CSD)-like phenomenon (Cutter and Huerter, 2007).

### SUMMARY OF THE INVENTION

Certain embodiments of the present invention relate to pharmaceutical compositions, such as compositions comprising a pharmacological activator of the PKG pathway in an amount effective for treating or preventing one or both of neural anoxia and reperfusion injury. In further embodiments, the pharmaceutical compositions further comprise an excipient.

In certain embodiments, the pharmacological activator of the PKG pathway may be used to provide neural anoxia protection. In such embodiments wherein the pharmacological activator used to provide neural axonia protection or reperfusion protection, the protection may result from an increase in potassium ion channel conductances.

In embodiments of the present invention wherein a pharmacological activator is contemplated, the activator may be any activator. In specific embodiments, the activator is an activator of the PKG pathway. Still further, the activator may be a PKG activator. In such embodiments the PKG activator may include 8-bromo-PET-cyclic GMPS; 8-pCPT-cylic GMPS,TEA; 8-Br-cGMPS, Na; or a combination thereof.

In other embodiments the pharmacological activator is a cGMP-specific agonist.

In further embodiments wherein a pharmacological activator is contemplated, the activator may activate K+ ion channel function.

In still further embodiments, wherein a pharmacological activator is contemplated, the activator may be a protein phosphatase activator. Still further, the protein phosphatise activator may be selected from the group consisting of PTPA.

In other embodiments wherein a pharmacological activator is contemplated, the activator is a PDE inhibitor selected from the group consisting of sildenafil citrate.

In certain embodiments, the pharmaceutical compositions described above may be used to treat or prevent a medical condition selected from the group consisting of damage from environmental hypoxia, spinal cord injury, and stroke.

Still other embodiments of the invention concern a pharmaceutical composition comprising: a nucleic acid encoding a pharmacological activator of the PKG pathway in an amount effective for treating or preventing one or both of neural anoxia and reperfusion injury.

Other aspects of the invention concern a method of treating or preventing a medical condition in a patient comprising administering to the patient a therapeutically effective amount of a pharmacological activator of the PKG pathway, wherein the medical condition is selected from the group consisting of damage from environmental hypoxia, spinal cord injury, stroke or a combination thereof.

Still further, in embodiments of the invention concerning a method of treating or preventing a medical condition in a patient comprising administering to the patient a therapeutically effective amount of a pharmacological activator of the PKG pathway, the PKG pathway is selected from the group consisting of a PKG activator; a cGMP-specific agonist; an activator of K+ ion channel function; a protein phosphatase activator; a sGC activator or a combination thereof. Still further the pharmacological activator is selected from the group consisting of 8-bromo-PET-cyclic GMPS; 8-pCPT-cylic GMPS,TEA; 8-Br-cGMPS, Na; PTPA; Sildenafil Citrate or a combination thereof.

Other embodiments of the invention concern a method of providing one or both of neural anoxia and reperfusion injury protection in a patient comprising administering to the patient a therapeutically effective amount of a pharmacological activator of the PKG pathway. In such embodiments, the neural anoxia or reperfusion injury protection results from an increase in potassium ion channel conductances.

In embodiments concerning providing neural anoxia protection, the method may include providing at least an effect selected from the group consisting of lowering the level of oxygen at which neural function becomes abnormal; decreasing the amount of cell death from lack of oxygen and reperfusion or a combination thereof.

Additional methods of the invention concern a method of treating or preventing a medical condition in a patient comprising administering to the patient a therapeutically effective amount of a composition comprising: a nucleic acid encoding a pharmacological activator of the PKG pathway in an amount effective for treating or preventing one or both of neural anoxia and reperfusion injury, wherein the medical condition is selected from the group consisting of damage from environmental hypoxia, spinal cord injury, and stroke.

### BRIEF DESCRIPTION OF THE DRAWINGS

Embodiments of the present invention will be described with reference to the accompanying drawings, wherein like reference numerals denote like parts and in which:

FIG. 1 is a graph showing animal survival after prolonged anoxic exposure is modulated by PKG activity by the foraging gene. Animals that genetically express increased PKG activity (+PKG) demonstrate an increase in animal survival after 6 hours of anoxia. Alternatively, animals that genetically express reduced PKG activity (−PKG) demonstrate increased death. Adult 5-9 day old Drosophila were exposed to 6 hours of anoxia, and allowed to recover for 24 hours. Number of flies that survived are depicted here, where Rovers (+PKG; N=29) demonstrate a significant survival rate over sitters (N=29) and fors2 (−PKG; N=28). Vertical bar chart is shown as mean±s.e.m., Letters A,B signify significant differences p<0.05, These were analyzed using a Two-Way ANOVA, F(2, 117)=12.693, p<0.001; SNK, p<0.05.

FIG. 2 is a graph showing turtle neuron cell death in response to pharmacologically activating (+PKG) or inhibiting (−PKG) the PKG pathway. Stimulation of the PKG pathway (+PKG) leads to an increase in cell survival during anoxia or reoxygenation. Alternatively, inhibition of the PKG pathway (−PKG) leads to increased cell death. N=Normoxia, A=Anoxia (4 hrs), R=Reoxygenation (4 hrs). Letters A,B signify significant differences p<0.05.

FIG. 3 is a pair of graphs showing anoxic coma onset is modulated by PKG activity through natural variation of the foraging gene. a, Time to anoxic coma onset (time to failure) in adult Drosophila melonogaster during acute hypoxia by air displacement using pure argon gas. b, PKG activity was assayed from the heads of animals in a. All vertical bar charts are shown as mean±s.e.m. Significant differences were established with p<0.05, where letters that differ on the graphs signify statistical groupings.

FIG. 4 is a pair of graphs showing to vivo pharmacological manipulation of various molecular targets in the PKG pathway modulates time to anoxic coma onset during acute hypoxia. a, Various volatilized pharmacological agents were used in vivo on the intermediate resilient allele sitter, to determine how different molecular targets modulate anoxic coma onset during acute hypoxia. b, PKG enzyme activity assays on heads of animals derived from experiments shown in a shows that agents which could manipulate PKG either directly such as 8-Bromo cGMP/[+]PKG and KT5823/[−]PKG or indirectly such as T0156/[−]PDE5/6 (which would increase intracellular cGMP) demonstrated significant effects compared to that of the sham control (N=6 for each treatment; One-Way ANOVA, F_{(5,30)}=20.898, p<0.001; SNK, p<0.05). However, targets that were downstream of PKG (see FIG. 4a), such as Cantharidin/[−]PP2A and DCA/[+]K⁺ channels, showed no significant effects on PKG enzyme activity levels compared to sham controls (SNK, p>0.05). All vertical bar charts are shown as mean±s.e.m. Significant differences were established with p<0.05, where letters that differ on the graphs signify statistical groupings. Horizontal dotted line represents mean of sham control for ease of comparison across treatments.

FIG. 5 is a schematic illustration of combinations of pharmacological agents that modulate time to anoxic coma onset during acute hypoxia reveal downstream and upstream molecular targets in the PKG pathway and a graph showing results from a test for resilience to anoxic coma onset during acute hypoxia. Adapted from Zhou et al. (1996), this diagram and experimental design represents upstream and downstream intracellular targets for manipulating the PKG pathway partially implicated in the modulation of hyperthermic stress (Dawson-Scully and Meldrum Robertson, 1998; Zhou et al., 1996) Protein phosphatase 2A (PP2A), cGMP-dependent protein kinase (PKG), cyclic GMP (cGMP), phosphodiesterases (PDE), and K⁺ channels are shown as potential targets for pharmacological manipulation. Inhibitory compounds are shown in red with a minus (−) sign, while activators are shown in green with a plus (+) sign. The diagram shows that molecular targets and pharmacological compounds to the left are downstream of those on the right, as shown by the large double arrow at the top of the diagram. Our hypothesis states that inhibition of this pathway would result in a decrease in whole cell K⁺ channel conductance, thereby leading to increased resilience to anoxic coma onset, b, Combinations of the pharmacological agents used, in vivo, shown in FIG. 4, were administered to adult Drosophila melanogaster, and then the animals were tested for resilience to anoxic coma onset during acute hypoxia. Vertical bar chart is shown as mean±s.e.m. Significant differences were established with p<0.05, where letters that differ on the graphs signify statistical groupings. Horizontal dotted line represents mean of sham control for ease of comparison across treatments.

FIG. 6 is a schematic illustration of in vivo treatment of adult Drosophila melanogaster with pharmacological agents and an anoxia stress assay.

### DETAILED DESCRIPTION OF THE PREFERRED EMBODIMENTS

During the acute phase shortly after the onset of an ischemic stroke, tissue in the penumbra surrounding an infarct receives sufficient blood flow to survive, but not enough to function. As time passes, neurons in this penumbra die (Baron and Moseley, 2000). The majority of cell death occurs in the brain at the time of medical treatment when the clot is removed and blood reperfusion occurs allowing return to normal oxygen levels. The inventors of the subject invention have surprisingly and unexpectedly discovered that activation of cGMP-dependent protein kinase (PKG) pathway mediates protection in nervous systems under anoxic stress and during reperfusion. As shown in the Examples, both genetic and pharmacological manipulations performed by the inventors demonstrate that there is a strong relationship between PKG activity and anoxic/reperfusion tolerance.

The pharmacological intervention of the present invention provides immediate and significant protection to neurons enduring anoxic stress. The invention involves the use of pharmacological activators of the cGMP-dependent protein kinase (PKG) which regulates potassium channel conductances.

Within the context of the present invention, neural anoxic protection means providing one or more of the following effects: (i) decreasing oxygen at which neural cell death begins to occur; and/or (ii) increasing the time for neural survival when exposed to anoxia;

Increasing the time of neural survival when anoxia is normalized during a stroke, but oxygen reperfusion occurs.

This invention provides protection as great as the known slow-acting treatments, and may provide more complete neural anoxic protection. The composition of the present invention may have one or more advantage, as compared to known treatments. A non limiting advantage is that the rapid protection provided by PKG activation is as great as that provided by slow-acting preconditioning treatments.

The present invention involves a neural protective composition during anoxia or reperfusion injury that includes a pharmacological activator of the PKG pathway. Any suitable pharmacological activator of the PKG pathway can be used.

A number of pharmacological activators of the PKG pathway are known; these activators effect different points in the enzyme pathway.

A pharmacological activitor of the PKG pathway suitable for the present invention, may be a PKG activator, for example: 8-bromo-PET-cyclic GMPS (Guanosine ‘ [symbol] ’-cyclic Monophosphorothioate); 8-Br-cGMPS, Na (Guanosine 3′,5′-cyclic Monophosphorothioate, 8-Bromo-, Sodium Salt);

These PKG pathway activators are commercially available; a leading supplier is Calbiochem (EMD) Biosciences, and Sigma Aldrich.

The pharmacological activator may be a cGMP-specific agonist. I.e. a suitable agonist might stimulate the activity of PDE5 or PDE9 in the breakdown of cGMP such as sildenafil citrate (Viagra).

The pharmacological activator may be an activator of ion channel function. The pharmacological activator may be a protein phosphatase activator. Suitable protein phosphatase activators include, for example, PTPA.

In other embodiments, a composition as described herein for protecting brain cells (e.g., neurons) from anoxia includes a nucleic acid encoding an activator of the PKG pathway.

It will be recognized by those of skill in the an that certain of the aforementioned activators are known in certain forms to have toxic effects, which must be addressed through formulation of the protective composition, as is known to those of skill in the art.

The neural anoxia and reperfusion injury protective composition of the present invention is suitable for treating a number of medical conditions including environmental hypoxia, spinal cord injury, stroke and migraine.

The manner of administering the neural anoxia and reperfusion injury protective composition of the present invention to a patient is not specifically restricted, and various methods will be readily apparent to persons skilled in the art. The neural anoxia and spreading depression protective composition, for example, could be delivered by inhalation, injection, or intravenously to a patient suffering from a stroke condition. The compound could also be taken orally. This would be particularly suitable where it is taken as a prophylactic.

Where the term “patient” is used in the present specification, it will be understood to include both human and non-human patients, including animals and plants.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates).

### Administration of Compositions

The compositions described herein may be administered to mammals (e.g., dog, cat, pig, horse, rodent, non-human primate, human) in any suitable formulation. For example, a composition including an activator of the PKG pathway or a nucleic acid encoding an activator of the PKG pathway may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions described herein may be administered to mammals by any conventional technique. Typically, such administration will be parenteral (e.g., intravenous, subcutaneous, intratumoral, intramuscular, intraperitoneal, or intrathecal introduction). The compositions may also be administered directly to a target site. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form. In therapeutic applications, the compositions described herein are administered to an individual already suffering from brain anoxia or reperfusion injury. In prophylactic applications, the compositions described herein are administered to an individual at risk of developing brain anoxia and/or reperfusion injury.

### Effective Doses

The compositions described herein are preferably administered to a mammal (e.g., dog, cat, pig, horse, rodent, non-human primate, human) in an effective amount, that is, an amount capable of producing a desirable result in a treated mammal (e.g., protecting brain cells such as neurons from anoxia and reperfusion injury). Such a therapeutically effective amount can be determined as described below.

Toxicity and therapeutic efficacy of the compositions described herein can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one subject depends on many factors, including the subject's size, body surface area, age, the particular composition to be administered, time and route of administration, general health, and other drugs being administered concurrently.

While this invention has been described with reference to illustrative embodiments and examples, the description is not intended to be construed in a limiting sense. Thus, various modifications of the illustrative embodiments, as well as other embodiments of the invention will be apparent to persons skilled in the art upon reference to this description. For example the modification of activators to improve solubility and cell permeability through packaging with modifying agents such as Membrane Translocation Proteins, esterification such that the esterified compound passes through lipid membranes and is converted into the active form in the cell by constitutive esterases, and the use of alternative salts of acid or basic compounds to improve solubility, stability, or buffer pH changes are apparent to persons skilled in the art upon reference to this description as means of improving stability, solubility, and delivery to the cell of the pharmacological activators.

All publications, patent and patent applications referred to herein are incorporated by reference in their entirety to the same extent as if each individual publication, patent or patent application was specifically and individually indicated to be incorporated by reference it its entirety.

### EXAMPLES

The present invention is further illustrated by the following specific examples. The examples are provided for illustration only and should not be construed as limiting the scope of the invention in any way.

### Example 1

### D. melanogaster Experiments

The foraging, (for) gene in Drosophila melanogaster encodes a cGMP-dependent protein kinase (PKG) (Osborne, K. A. et al., 1997). Animal survival after 24 hours recovery from 6 hours of complete anoxia for both high and low PKG activity strains (for^{R} and for^{s2} respectively).

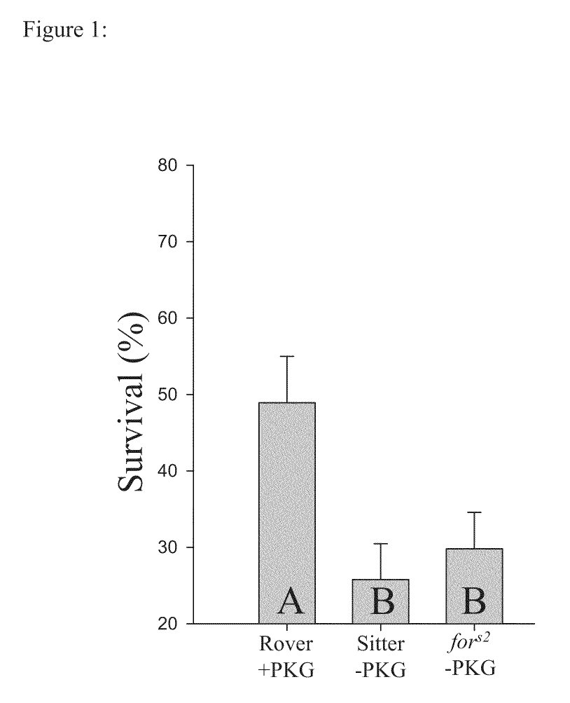

Animals that genetically express increased PKG activity (+PKG) demonstrate an increase in animal survival after 6 hours of anoxia. Alternatively, animals that genetically express reduced PKG activity (−PKG) demonstrate increased death. Adult 5-9 day old Drosophila, were exposed to 6 hours of anoxia, and allowed to recover for 24 hours. Number of flies that survived are depicted here, where Rovers (+PKG; N=29) demonstrate a significant survival rate over sitters (N=29) and fors2 (−PKG; N=28). Vertical bar chart is shown as mean s.e.m. Letters A,B signify significant differences p<0.05. These were analyzed using a Two-Way ANOVA, F(2, 117)=12.693, p<0.001; SNK, p<0.05, (Shown in FIG. 1)

Thus, genetic manipulations demonstrate that there is a positive relationship between PKG activity and prolonged anoxia tolerance for D. melanogaster.

D. melanogaster were reared on a yeast-sugar-agar medium at 24±1° C. under 12L:12D light cycle and aged to 5-9 days old. We tested animal survival in prolonged anoxia, where we exposed adult flies with varying PKG activity (Rover-high PKG, sitter and fors2=low) to six hours of anoxia and subsequent reoxygenation. We found that after 24 hours of recovery, rovers exhibit a significant increase in the number of animals that survive compared with sitters and fors2 strains.

### Example 2

### Trachemys scripta Experiments

To induce anoxia, turtles were placed individually in a tightly sealed container at room temperature (22-23° C.) with positive pressure flow-through nitrogen gas (99.99% N₂, Air Gas, Miami, Fla., USA) for the experimental time period. Animals subjected to reoxygenation were allowed to recover in room air. Control cultured neurons were utilized directly from the aquaria. For the study of cell survival, turtles cultured neurons subjected to anoxia for 0 h and 4 h anoxia. A separate group was exposed to 4 h anoxia followed by 4 h of reoxygenation. 4 h of reoxygenation of the anoxic cells was also performed (simulates reperfusion injury from stroke). Propidium iodide was added to the cell culture medium after each treatment (N=normoxia, A=4 hours of anoxia and R=4 hours of reoxygenation). For each circumstance three treatments were used (1: no drug; 2: PKG activator; 3: PKG Inhibitor).

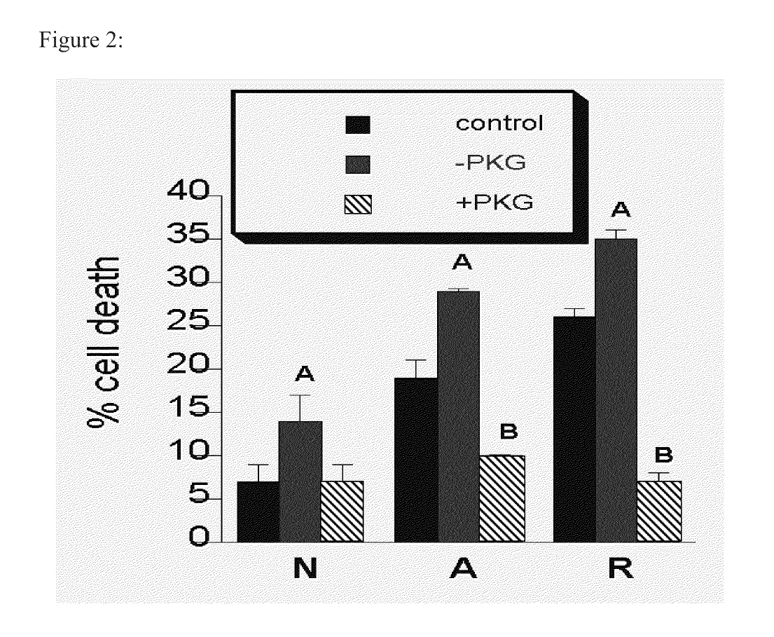

Turtle neuron cell death in response to pharmacologically activating (+PKG) or inhibiting (−PKG) the PKG pathway. Stimulation of the PKG pathway (+PKG) leads to an increase in cell survival during anoxia or reoxygenation. Alternatively, inhibition of the PKG pathway (−PKG) leads to increased cell death. N=Normoxia, A=Anoxia. (4 hrs), R=Reoxygenation (4 hrs), Letters A,B signify significant differences p<0.05, (Shown in FIG. 2).

Thus pharmacological activation of PKG results in no damage from anoxic or reperfusion injury stress when compared to controls.

### Example 3

### Controlling Anoxic Tolerance in Adult Drosophila via the cGMP-PKG Pathway

In the experiments described herein, a cGMP dependent kinase (PKG) cascade was identified as a biochemical pathway critical for controlling low-oxygen tolerance in the adult fruit fly, Drosophila melanogaster. Even though adult Drosophila can survive in 0% oxygen (anoxia) environments for hours, air with less than 2% oxygen rapidly induces locomotory failure resulting in an anoxic coma. Natural genetic variation and an induced mutation in the foraging (for) gene, which encodes a Drosophila PKG, were used to demonstrate that the onset of anoxic coma is correlated with PKG activity. Flies that have lower PKG activity demonstrate a significant increase in time to the onset of anoxic coma. Further, in vivo pharmacological manipulations reveal that reducing either PKG or protein phosphatase 2A (PP2A) activity increases tolerance of behavior to acute hypoxic conditions. Alternatively, PKG activation and Phosphodiesterase (PDE5/6) inhibition significantly reduce the time to the onset of anoxic coma. By manipulating these targets in paired combinations, a specific PKG cascade, with upstream and downstream components, was characterized. Further, using genetic variants of PKG expression/activity subjected to chronic anoxia over 6 hours, ˜50% of animals with higher PKG activity survive, while only ˜25% of those with lower PKG activity survive after a 24 hour recovery. Therefore, in the experiments described herein, the PKG pathway and the differential protection of function vs. survival in a critically low oxygen environment is described.

It was hypothesized that natural variation in for would modulate acute low-oxygen tolerance during locomotory behavior and animal survival in adult Drosophila melanogaster. Herein, it was demonstrated for the first time that natural variation in tolerance of behavior to acute hypoxia between the rover and sitter alleles of the foraging gene, involves components of the PKG pathway in Drosophila melanogaster; and is inversely related to the tolerance of survival during prolonged anoxia.

### Methods and Materials

Using both genetics and pharmacology, the roles and order of function of additional cGMP-dependent signaling molecules in modulating acute low-oxygen sensitivity of locomotory failure were examined. To do this, we imposed acute hypoxia (<0.2% O₂ within 5 min) by displacing environmental oxygen with the inert gas argon and then measured time to failure of locomotion (anoxic coma), which can be observed in less than 2% atmospheric oxygen (Haddad, 2000). We also used a novel but reliable method of pharmacological volatilization as a means to manipulate, in vivo, the activity of target enzymes and molecules such as PKG. Flies were frozen immediately after each trial, and heads were dissected and examined for PKG activity, as an indicator for activity levels in the bruin (Belay et al., 2007; Kaun et al., 2007b). Lastly, using adult flies we tested what has been previously shown in embryos and larva (Wingrove and O'Farrell, 1999), the effect of prolonged anoxia (6 hours) on animal survival, after a 24 hour recovery.

Two naturally occurring strains of adult Drosophila melanogaster were used in this study; a rover strain homozygous for the for^{R} allele (high PKG activity), and a sitter strain, homozygous for^{s} (low PKG activity). These strains are isogenized natural polymorphisms of the foraging gene, located on the second chromosome (Fitzpatrick et al., 2007; Sokolowski, 1980). Additionally, the sitter mutant strain for^{s2} which was previously generated in the laboratory was also utilized. This strain has a rover genetic background with a mutation at for leading to PKG activity/transcript levels lower than that observed in sitters (Pereira and Sokolowski, 1993). All flies were reared in the same fashion; 12 h light: 12 h dark light cycle with lights on at 0800 h, equal density (approximately 100 flies in a 170 mL plastic culture bottle containing 40 mL of a standard yeast-sucrose-agar medium), and same age (5-9 day old adults at testing time) in an incubator at 25° C. Flies that were used in this study were not exposed to anesthesia for at least 24 hours before trials.

Ten flies (separated into males or females) from each of the three strains (rover [for^{R}], sitter [for^{s}] and for^{s2}) were selected and placed in three separate vials with food at least 24 hours before each experiment. At the time of the experiment, flies were emptied into 1.0 mL empty test vials covered with permeable sponge caps of equivalent thickness, allowing for consistent gas exchange. These test vials were then placed into a 600 mL beaker which was covered with parafilm, creating a closed chamber with an escape hole in the parafilm (1 cm diameter). The purpose of this hole is to allow the lighter normal air (compared to argon which is heavier) to escape from the top of the beaker. The parafilm top was punctured with a needle connected to a gas tank (by a plastic hose), through which the inert gas argon was expelled. Preliminary work was done with pure nitrogen, where no significant differences between the uses of the two gases were observed (data not shown). Argon gas was chosen to displace normoxic air over nitrogen in these studies since previous work demonstrated that nitrogen treated Drosophila had an unusually prolonged recovery time from the onset of chill coma (Nilson et al., 2006).

The needle tip was fastened to the bottom of the beaker and inserted into a larger sponge of similar texture to the vial sponge caps to ensure that gas expelled from the needle was dispersed equally throughout the beaker. Also, to ensure that all test vial positions in the beaker received equal gas flow, test vial positions were alternated every trial to reduce variability.

The experiment required exposing the test vials with 10 flies in a vial within the container to pure argon gas expelled from the tank at a flow rate of 600 cc/min±5%. Time of behavioral failure was recorded by observing the fly undergo a seizure, which was quickly followed by an anoxic coma. A novel computer program entitled Multi-Arena-Multi-Event-Recorder (MAMER, freely available at cfly.utm.utoronto.ca/MAMER), developed by Dr. Craig A. L. Riedl, was used to record neural failure time for each individual fly. This was accomplished by starting the timer on the program at the initiation of gas flow. The observer would then type “1” if a fly failed in vial 1 (where 10 flies reside), “2” for fly failure in vial 2, and “3” for observed failure in vial 3. The program recorded these times of failure for each individual fly within the vials and then computed an average failure time for each vial (each vial has 10 flies but gives an average value for N=1). The averaged failure times represent the N data in the figures, and successfully distinguished failure times among the different strains. Upon failure of all flies, the vials were placed in a freezer at 20° C. to preserve PKG levels at time of behavioral failure. These flies were later subjected to PKG enzyme activity assays to measure PKG enzyme levels. Males and females were tested separately, and no significant differences were found between any of the

Ten the (separated into males or females) from each of the three strains (rover [for^{R}], sitter [for^{s}] and for^{s2}) were selected and placed in three separate vials with food at least 24 hours before each experiment. These test vials were then placed into a 1000 mL beaker which was covered with parafilm, creating a closed chamber with an escape hole (1 cm diameter). The parafilm top was punctured with a needle connected to a gas tank (by a plastic hose), through which the inert gas argon was expelled.

The needle tip was fastened to the bottom of the beaker and inserted into a larger sponge of similar texture to the vial sponge caps to ensure that gas expelled from the needle was dispersed equally throughout the beaker. The gas was turned on at a flow of 600 cc/min and left for 6 hours, Vials were removed after the experiment and placed in to a 25° C. 12 h dark 12 h light cycle for 24 hours, and animals alive were counted.

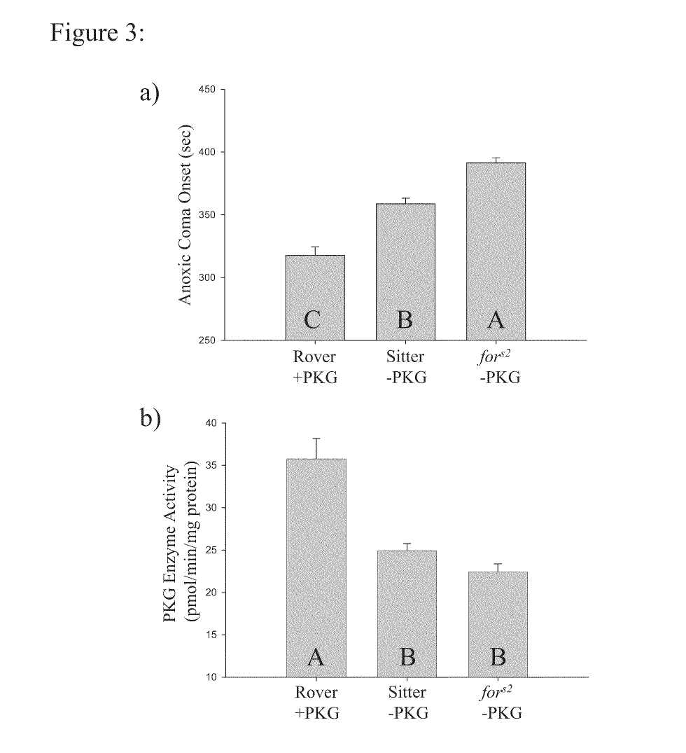

The same behavioral assay described above was employed; however, flies were pre-treated with chemical agents to observe their effects on tolerance to anoxic stress. The pharmacological behavioral assays were conducted on the sitter strain because of its intermediate tolerance when compared to rovers and the sitter mutant (FIG. 3). Adult sitters were exposed to various drugs predicted to have an effect on targets involved in the PKG pathway. These drugs included (from Sigma Aldrich): 10 mM T0156 (a cGMP-specific phosphodiesterase-5 inhibitor), 10 mM 8-Bromo-cGMP PKG activator), 1 mM KT5823 (a PKG inhibitor), 1 mM Cantharidin (a PP2A inhibitor), and 200 mM DCA (a K⁺ channel activator). All drugs were solubilized in dimethyl sulfoxide (DMSO), where flies treated with only DMSO were used as a sham controls. We developed a novel assay to administer these drugs to whole adults in vivo through volatilization, since we used concentrations of the drug at 1000-fold (uM vs. mM) concentrations used in vitro (Dawson-Scully et al., 2007), 10 μL of drug solution was applied to a crushed Kim wipe (VWR International) at the bottom of each test vial. An additional Kim wipe was crushed over top of this to prevent direct contact of the fly on the solution. 10 flies were then placed in the vial which was capped with a semi-permeable flug and covered with a cut-out finger of a large latex glove to prevent chemical vapours from escaping in the dark. The flies were subjected to the drug for 1 hr prior to each behavioral assay.

Drug combinations were employed to observe the effects of activating and/or inhibiting various participants in the PKG pathway simultaneously to determine downstream and upstream targets. Here 20 uL of DMSO was used as a sham control, and combinations of two pharmacological treatments were added as two separate 10 uL aliquots. The same drugs and protocol described above were used in this assay. The drug combinations tested were: 10 mM 8-Bromo-cGMP/1 mM KT5823, 10 mM 8-Bromo-cGMP/1 mM Cantharidin, 10 mM 8-Bromo-cGMP/10 mM T0156, 10 mM 8-Bromo-cGMP/200 mM DCA, 1 mM KT5823/1 mM Cantharidin, 1 mM KT5823/10 mM T0156, 1 mM KT5823/200 mM DCA, 1 mM Cantharidin/10 mM T0156, 1 mM Cantharidin/200 mM DCA and 10 mM T0156/200 mM DCA.

PKG enzyme activity assays were conducted according to the procedure outlined in Kaun et al. (2007) (Kaun et al., 2007b). Adult Drosophila were decapitated and the heads were homogenized in 25 mM 1-1 Tris (pH 7.4), 1 mM EDTA, 2 mM 1-1 EGTA, 5 mM 1-1 β-mercaptoethanol, 0.05% Triton X-100 and protease inhibitor solution (Roche Diagnostics). Following microcentrifugation for 5 min, the supernatant was removed and those supernatants containing equal amounts of total protein were examined for PKG enzyme activity. The reaction mixture contained the following substances: 40 mM 1-1 Tris-HCl (pH 7.4), 20 mM 1-1 magnesium acetate, 0.2 mM 1-1 [γ³²P]ATP (500-1000 c.p.m. pmol-1), 113 mg ml-1 heptapeptide (RKRSRAE), 3 mM 1-1 cGMP and a highly specific inhibitor of cAMP-dependent protein kinase. The next step of the procedure involved incubating the reaction mixtures at a temperature of 30° C. for 10 min, followed by ending the reaction by spotting 70 μl of the reaction mixture onto Whatman P-81 filters. To remove any unreacted [γ³²P]ATP, these spots were then soaked with 75 mM 1-1 H₃PO₄ for 5 min and washed three times with 75 mM 1-1 H₃PO₄. Before quantifying enzyme activity, filters were rinsed with 100% ethanol and air dried. To calculate PKG enzyme activity, counts were taken in a Wallac 1409 Liquid Scintillation Counter using universal scintillation cocktail (ICN). PKG activity was presented in the figures as pmol of ³²P incorporated into the substrate min-1 mg-1 protein.

Data were analyzed using One-Way and Two-Way ANOVA followed by a post-hoc Multiple Comparisons test (SNK=Student-Neuman-Keul's test). In cases where normality or equal variance failed, non parametric tests on the ANOVA on ranks were used. Significant differences were established with p<0.05, where letters that differed on the graphs signified statistical groupings. In behavioral trials, N=1 represents a trial which consisted of one vial with 10 adult flies.

### Results

To investigate the role of PKG in regulating sensitivity to acute hypoxia leading to anoxic coma, homozygous flies with different for alleles, either the natural rover (higher PKG activity) or sitter (lower PKG activity) alleles, or for^{s2}, a hypomorphic foraging mutant induced on a rover genetic background, were assayed for locomotion failure (see Methods). Time to anoxic coma onset (time to failure) in adult Drosophila melonogaster during acute hypoxia by air displacement using pure argon gas, was significantly increased in the natural allele of the foraging gene, sitter (N=18; low PKG activity), when compared with rover (N=18; high PKG activity; FIG. 3a). Further, for^{s3} (N=18), foraging mutant in rover (low PKG activity), was also significantly resilient to acute hypoxia when compared to the two natural alleles (One-Way ANOVA, F_{(2,50)}=50.352, p<0.001; Multiple Comparisons, SNK, p<0.05). PKG activity was then assayed from the heads of animals in FIG. 3a, confirming that rovers exhibited high PKG activity, whereas sitters and for^{s2} showed significantly lower PKG activity (N=6 for each genotype; One-Way ANOVA, F_{(2,15)}=20.360, p<0.001; SNK, p<0.05; FIG. 3b).

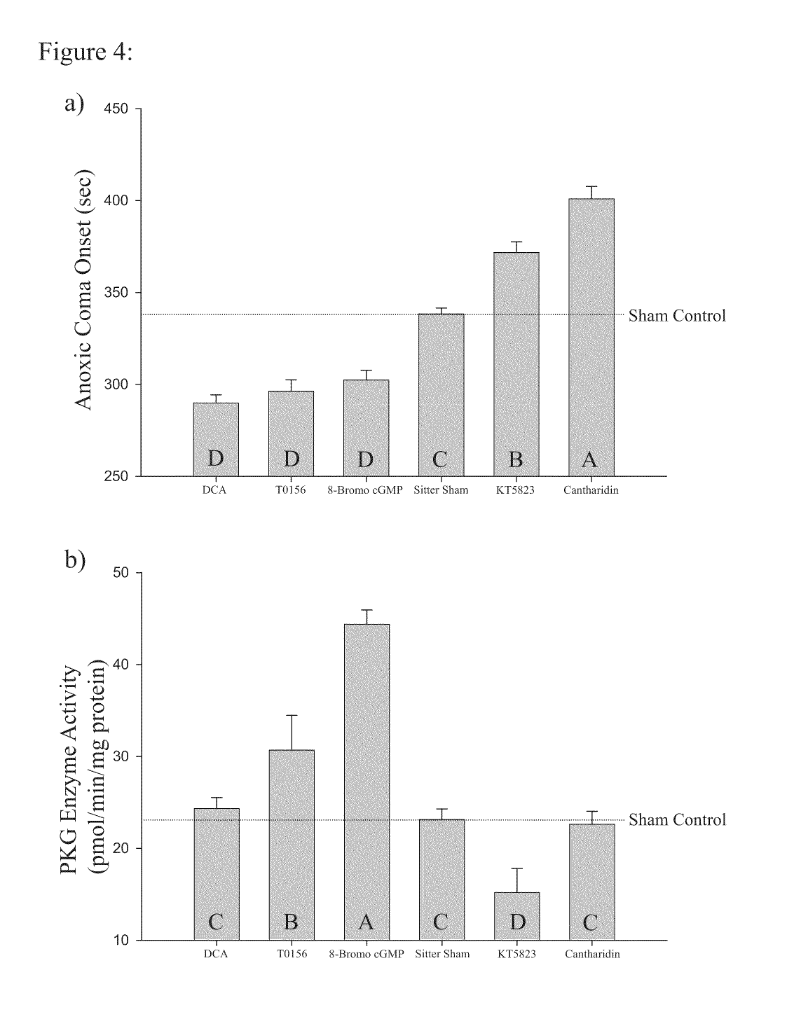

We assessed whether fly PKG could be manipulated in vivo by means of using the volatilization of pharmacological agents. Previously we have used pharmacology on tissue preparations to demonstrate that cGMP, PKG, and PP2A targets regulate the tolerance of synaptic transmission during acute hyperthermia at the Drosophila larval neuromuscular junction (Dawson-Scully et al., 2007). In the present study, we examine the rote of these targets in vivo using adult sitter flies by depositing pharmacological agents dissolved in dimethyl sulfoxide (DMSO) on a cellulose tissue, and allowing them to volatize over minutes in an air tight vial at room temperature (FIG. 4). In order to examine the PKG-PP2A-K⁺ channel axis, we used Dichloroacetate (DCA), a compound that has been shown to augment the nitric oxide (NO)/K⁺ channel axis (Michelakis et al., 2003) in a number of cell types (Bonnet et al., 2007; Michelakis et al., 2002). Interestingly, previous work has also shown that, physiologically, neuronal whole-cell K⁺ currents are reduced in animals that have undergone a heat shock preconditioning (Ramirez et al., 1999). This is associated with the protection of the nervous system during hyperthermic stress (Dawson-Scully and Meldrum Robertson, 1998). We therefore tested the hypothesis that DCA, which is known to increase K⁺ currents, will induce sensitivity of behavior to acute anoxia (Bonnet et al., 2007; Michelakis et al., 2003). We found that each treatment had a significant effect, either increasing or decreasing anoxic coma onset sensitivity (Kruskal-Wallis, H₍₅₎=107.454, p<0.001; Multiple Comparisons, Dunn's, p<0.05; FIG. 4a), where 1 mM KT5823/[−]PKG (N=47; pharmacological agent/[+ activates/− inhibits]target) and 1 mM Cantharidin/−PP2A (N=12) demonstrated significant resilience to anoxic coma (Dunn's, p<0.05) and 200 mM DCA/[+]K⁺ channels (N=15), 10 mM 8-Bromo cGMP/[+]PKG (N=34), and 10 mM T-0156/[−]PDE5/6 (N=16) exhibited significant sensitivity to anoxic coma (Dunn's, p<0.05).

PKG enzyme activity assays on heads of animals derived from experiments shown in FIG. 4a. shows that agents which could manipulate PKG activity either directly such as 8-Bromo cGMP/[+]PKG and KT5823/[−]PKG or indirectly such as T0156/[−]PDE5/6 (which would increase intracellular cGMP) demonstrated significant effects compared to that of the sham control (N=6 for each treatment; One-Way ANOVA, F_{(5,30)}=20.898, p<0.001; SNK, p<0.05; FIG. 4b). However, targets that were downstream of PKG (sec FIG. 4a), such as Cantharidin/[−]PP2A and DCA/[+]K⁺ channels, showed no significant effects on PKG enzyme activity levels compared to sham controls (SNK, p>0.05).

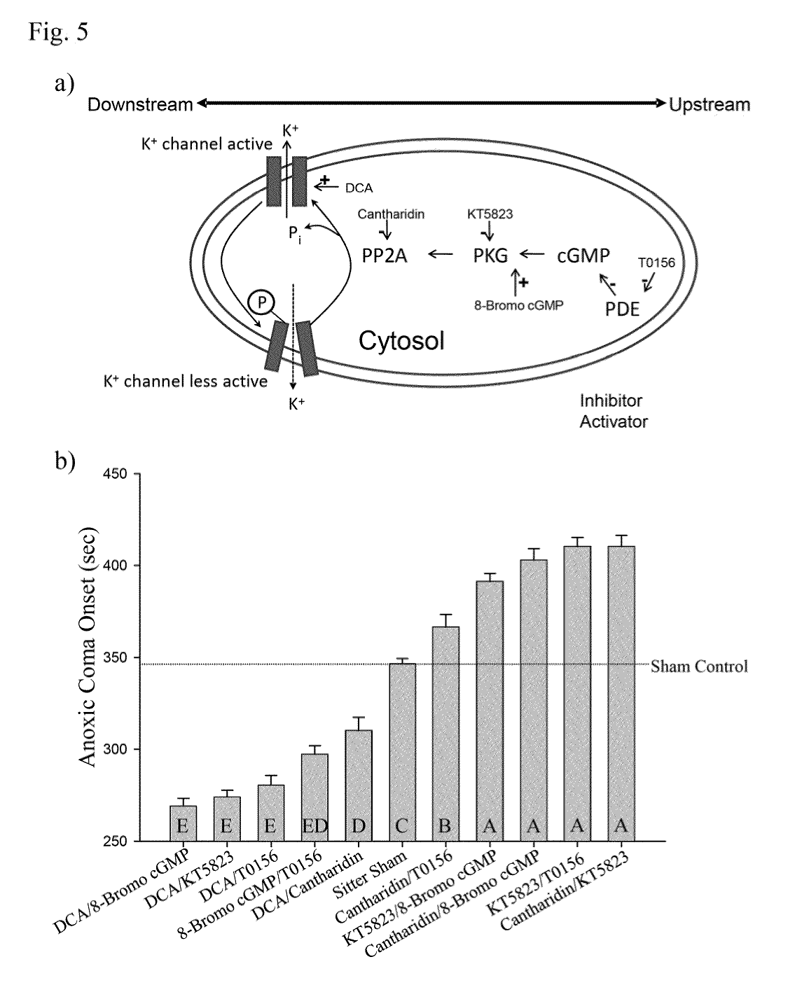

We next used two simultaneous pharmacological treatments of the above mentioned compounds in all complementary combinations, to verify upstream and downstream components (FIG. 5a) of this proposed pathway. We investigated the hypothesis that the effects of downstream molecular targets would override upstream targets. Similar to experiments using individual compounds (FIG. 4a), we found that each combined treatment of two agents either significantly increased or decreased anoxic coma onset sensitivity when compared to sham controls (One-Way ANOVA, F_{(10,184)}=105.634, p<0.001; SINK, p<0.05). As predicted, we found that pharmacological agents that inhibited or activated downstream molecular targets, as shown in FIG. 5a, directed the anoxic coma onset phenotype. For example, anytime animals (FIG. 5b) were treated with a combination of drugs that included DCA, a significant decrease in resilience to anoxic coma onset was observed, when compared to sham controls (SNK, p<0.05).

We tested animal survival in prolonged anoxia, where we exposed adult flies with varying PKG activity (Rover=high PKG, sitter and for^{s2}=low) to six hours of anoxia and subsequent reoxygenation. We found that after 24 hours of recovery, rovers exhibit a significant increase in the number of animals that survive compared with sitters and for^{s2} strains (FIG. 1).

Not all animals are equally susceptible to critically low oxygen. Facultative anaerobes are evolutionarily adapted to withstand long periods without oxygen; anoxia survival tolerance of at least several hours has been established in the fruit fly Drosophila melanogaster (Haddad, 2006; Wingrove and O'Farrell, 1999) while some turtles can withstand anoxia for days to months (Ultsch, 2006). These anoxia tolerant organisms, in contrast to mammalian systems, enter a state of deep reversible hypometabolism, thereby losing neural function but maintaining a balance between energy requirements and supply by suppressing energy demanding functions, including the release of excitatory neurotransmitters (Milton and Lutz, 2005; Milton et al., 2002) and ion flux (Bickler et al., 2000; Perez-Pinzon et al., 1992; Sick et al., 1982), which together suppress electrical activity (Fernandes et al., 1997; Gu and Haddad, 1999). Anoxia tolerance then permits survival of extended anoxia without neuronal deficit (Haddad, 2006; Kesaraju et al., 2009). In the work reported here, we demonstrate that under anoxic stress we have identified two opposing phenotypes: 1) During PKG pathway inhibition, we observe the protection of locomotion during acute hypoxia, and 2) During PKG pathway activation, we observe the protection of survival during prolonged anoxia.

Anoxic coma onset data presented here suggests that an inhibition in the PKG signaling cascade promotes increased behavioral tolerance to acute low-oxygen environments before anoxic coma occurs (FIG. 3). Further, the data suggest that this pathway also acts through PDEs and PP2A (FIG. 4). We propose that the natural polymorphism in the foraging gene is functionally relevant to the limits for low-oxygen stress tolerance in fly behavior. Thus, oxygen tolerance may have played a pivotal role in how the rover and sitter alleles were selected for during the evolution of this polymorphism. Further, since rovers and sitters differ in their ability to tolerate low-oxygen stress, ecological implications of habitat limitations and sudden environmental changes may contribute to changes in allelic frequencies in the wild. One example of low oxygen stress on fruit flies is drowning due to excessive rainfall, a variable environmental factor in any habitat. The foraging gene functions in food-related behaviors across diverse taxa (Reaume and Sokolowski, 2009). Whether for's function in oxygen tolerance is also conserved remains to be determined.

Our results identify a molecular pathway involved in the modulation of anoxic coma, a response to acute hypoxia. This work also raises the possibility that future studies may reveal polymorphisms in genes encoding molecular targets described here affecting risk to low-oxygen stress pathologies. Further, the finding that molecules important for regulating the tolerance to acute hypoxia also function similarly during hyperthermic stress (Dawson-Scully et al., 2007) suggests that a conserved mechanism subserves both types of stress. At the level of the nervous system, these types of stresses may act to deplete cellular energy, due to hyperactivity during hyperthermia or blockage of cellular metabolism during hypoxia (Carling, 2004). At the cellular level, the reduction of whole-cell K⁺ current may be a form of cellular energy conservation (Weckstrom and Laughlin, 1995) that confers tolerance to such stresses (Ramirez et al., 1999).

Recent published work in non-anoxia tolerant systems has demonstrated loosely that PKG activation is protective for cell survival during anoxia in mammals (Caretti et al., 2008). However, little is known about how this protection relates to the tolerance of neural function, and what components of the PKG pathway are involved. Through the use of the fruit fly, an anoxia tolerant organism, we have demonstrated that the cGMP-PKG-PP2A pathway alters behavioral tolerance to acute hypoxia and mediates neuroprotection (a term that now includes neural function and survival). In the results shown here using DCA, flies have similar coma onset to DCA application as they do with PKG activation (FIG. 4a). An interesting finding was that DCA did not effect PKG activity levels whatsoever (FIG. 4b). Therefore, it appears that the K⁺ current increase from DCA exposure may be direct or through another pathway other than the PKG pathway. In flies, the mammalian homolog of the SUR2 subunit of mK_{ATP} (dSUR) was also shown to play a protective role against hypoxic stress (Akasaka et al., 2006).

The data here, then, suggest that inhibition of the PKG/PP2A pathway (FIG. 3) extends neural function in the face of acute hypoxia. By contrast, an up-regulation of the PKG pathway increases cell survival; it is not yet known if this occurs by a more rapid suppression of function, such as what is shown in turtles (Milton et al., 2002). Under anoxic stress, anoxic tolerant animals such as turtles suppress metabolic requirements and enter a coma-like state. Therefore, it would be of interest to determine if fly coma onset is such a suppression. Our data suggest that the PKG pathway shows an inverse relationship between preserving function, but inducing cell death during anoxia. Therefore, activating the PKG pathway confers protection of survival, but reduces tolerance of neural function to acute hypoxia. Our current hypothesis is: inhibition of the PKG pathway leads to the protection of neural function during acute hypoxia, allowing for the nervous system to continue operating in the face of increased physiological stress. However, this protection comes at the cost of decreased survival: when the suppression of cell function is blocked, as with reduced PKG activity, mortality increases significantly. To elucidate these other pathways of interest for animal survival during chronic anoxia we are currently attempting to develop a reliable pharmacological assay that will last multiple hours.

Since our work demonstrates that the PKG pathway can be manipulated to protect either function or survival, there is the potential to rapidly intervene with a pharmacological treatment to differentially protect either function or survival potentially at the cellular level, depending on what is required during a stroke event.

### Example 4

### Additional Drosophila Experiments

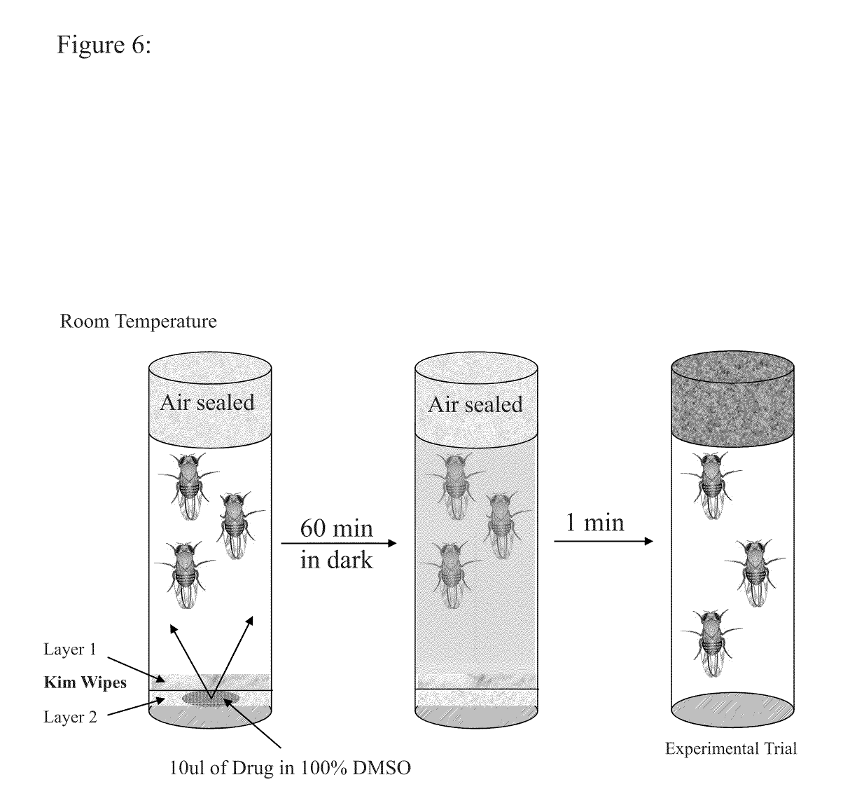

Referring to FIG. 6, this figure is a schematic illustration of in vivo treatment of adult Drosophila melanogaster with pharmacological agents and an anoxia stress assay. 5-9 day old adult Drosophila melanogaster are exposed to the pharmacological agents, volatilized from 10 uL of DMSO at room temperature for 60 minutes in the dark, and are tested for tolerances to anoxic coma onset. Adults are exposed to various drugs known to target components of the PKG pathway: 10 mM T0156, a cGMP-specific phosphodiesterase-5 inhibitor, 10 mM 8-Bromo-cGMP, a PKG activator, 1 mM KT5823, a PKG inhibitor, and 1 mM Cantharidin, a PP2A inhibitor. All drugs are solubilized dimethyl sulfoxide (DMSO), where flies treated with only DMSO are used as sham controls. Drugs are administered to whole adults in vivo through volatilization at concentrations 10-fold higher than those used in vitro (Dawson-Scully et al., 2007). Behavioral coma onset is then measured by time to failure with the exposure of argon to displace room air at 600 cc/minute.

### REFERENCES

Akasaka, T., Klinedinst, S., Ocorr, K., Bustamante, E. L., Kim, S. K. and Bodmer, R, (2006). The ATP-sensitive potassium (KATP) channel-encoded dSUR gene is required for Drosophila heart function and is regulated by tinman. Proc Natl Acad Sci USA 103, 11999-2004.
Belay, A. T., Scheiner, R., So, A. K., Douglas, S. J., Chakaborty-Chatterjee, M., Levine, J. D. and Sokolowski, M. B. (2007). The foraging gene of Drosophila melanogaster: spatial-expression analysis and sucrose responsiveness. J Comp Neurol 504, 570-82.
Ben-Shahar, Y., Robichon, A., Sokolowski, M. B. and Robinson, G. E. (2002). Influence of gene action across different time scales on behavior, Science 296, 741-4.
Bickler, P. F., Donohoe, P. H. and Buck, L. T. (2000). Hypoxia-induced silencing of NMDA receptors in turtle neurons. J Neurosci 20, 3522-8.
Bonnet, S., Archer, S. L., Allalunis-Turner, J., Haromy, A., Beaulieu, C., Thompson, R., C. T., Lopaschuk, G. D., Puttagunta, L., Harry, G. et al. (2007). A mitochondria-K+ channel axis is suppressed in cancer and its normalization promotes apoptosis and inhibits cancer growth. Cancer Cell 11, 37-51.
Canini, F., Bourdon, L., Cespuglio, R. and Buguet, A. (1997) Voltametric assessment of brain nitric oxide during heatstroke in rats. Neurosci. Lett., 231, 67-70.
Canini, F., Buguet, A. and Bourdon, L. (2001) Inhibition of different types of nitric oxide synthase: effect on thermoregulation in the rat exposed to high ambient temperature. Neurosci. Lett., 316, 45-49.
Caretti, A., Bianciardi, P., Ronchi, R., Fantacci, M., Guazzi, M. and Samaja, M. (2008). Phosphodiesterase-5 inhibition abolishes neuron apoptosis induced by chronic hypoxia independently of hypoxia-inducible factor-1alpha signaling. Exp Biol Med (Maywood) 233, 1222-30.
Carling, D. (2004). The AMP-activated protein kinase cascade—a unifying system for energy control. Trends Biochem Sci 29, 18-24.
Chai, Y. and Lin, Y. F. (2008). Dual regulation of the ATP-sensitive potassium channel by activation of cGMP-dependent protein kinase, Pflugers Arch 456, 897-915,
Chang, C. P., Lee, C. C., Chen, S. H. and (2004) Aminoguanidine protects against intracranial hypertension and cerebral ischemic injury in experimental heatstroke. J. Pharmacol. Sci., 95, 56-64.
Corbin, J. D. and Francis, S. H. (1999) Cyclic GMP Phosphodiesterase-5: Target of Sildenafil, J. Biol. Chem., 274, 13729-13732.
Dostmann, W. R., Tegge, W., Frank, R., Nickl, C. K., Taylor, M. S. and Brayden, J. E. (2002) Exploring the mechanisms of vascular smooth muscle tone with highly specific, membrane-permeable inhibitors of cyclic GMP-dependent protein kinase 1alpha. Pharmacol. Ther., 93, 203-215.
Dawson-Scully, K., Armstrong, G. A., Kent, C., Robertson, R. M. and Sokolowski, M. B. (2007), Natural variation in the thermotolerance of neural function and behavior due to a cGMP-dependent protein kinase. PLoS ONE 2, e773.
Dawson-Scully, K. and Meldrum Robertson, R. (1998). Heat shock protects synaptic transmission in flight motor circuitry of locusts. Neuroreport 9, 2589-93.
Downey, J. M., Krieg, T. and Cohen, M. V. (2008), Mapping preconditioning's signaling pathways: an engineering approach. Ann N Y Acad Sci 1123, 187-96,
Fernandes, J. A., Lutz, P. L., Tannenbaum, A., Todorov, A. T., Liebovitch, L. and Vertes, R. (1997). Electroencephalogram activity in the anoxic turtle brain, Am J Physiol 273, R911-9,
Fitzpatrick, M. J., Feder, E., Rowe, L. and Sokolowski, M. B. (2007). Maintaining a behaviour polymorphism by frequency-dependent selection on a single gene. Nature 447, 210-2.
Fitzpatrick, M. J. and Sokolowski, M. B. (2004) in Search of food: Exploring the evolutionary link between cGMP-dependent protein kinase (PKG) and behaviour, Integr. Comp. Biol., 44, 28-36.
Gerstberger, R. (1999) Nitric Oxide and Body Temperature Control. News Physiol. Sci., 14, 30-36.
Glazer, J. L. Am Fam Physician. 71; 11 2133 (2005),
Gu, X. Q. and Haddad, G. G. (1999), Drosophila neurons respond differently to hypoxia and cyanide than rat neurons. Brain Res 845, 6-13.
Gulec, G. and Noyan, B. (2001) Do recurrent febrile convulsions decrease the threshold for pilocarpine-induced seizures? Effects of nitric oxide, Brain Res. Dev. Brain Res., 126, 223-228.
Gurnett, A. et al., (20020 J. Biol. Chem. 277: 15913-15922.
Haddad, G. G. (2000). Enhancing our understanding of the molecular responses to hypoxia in mammals using Drosophila melanogaster, J Appl Physiol 88, 1481-7.
Haddad, G. G. (2006). Tolerance to low O2: lessons from invertebrate genetic models, Exp Physiol 91, 277-82.
Han, J., Kim, N., Kim, E., Ho, W. K. and Earm, Y. E. (2001). Modulation of ATP-sensitive potassium channels by cGMP-dependent protein kinase in rabbit ventricular myocytes. J Biol Chem 276, 22140-7.
Jayakar, S. S. and Dikshit, M. (2004) AMPA receptor regulation mechanisms: future target for safer neuroprotective drugs. Int. J. Neurosci., 114, 695-734,
Kaun, K. R., Hendel, T., Gerber. B. and Sokolowski, M. B. (2007a). Natural variation in Drosophila larval reward learning and memory due to a cGMP-dependent protein kinase. Learn Mem 14, 342-9.
Kaun, K. R., Riedl, C. A., Chakaborty-Chatterjee, M., Belay, A. T., Douglas, S. J., Gibbs, A. G. and Sokolowski, M. B. (2007b). Natural variation in food acquisition mediated via a Drosophila cGMP-dependent protein kinase. J Exp Biol 210, 3547-58.
Kesaraju, S., Schmidt-Kastrier, R., Prentice, H. M. and Milton, S. L. (2009). Modulation of stress proteins and apoptotic regulators in the anoxia tolerant turtle brain. J Neurochem 109, 1413-26.
Klyueva, Y. A., Bashkatova, V. G., Vitskova, G. Y., Narkevich, V. B., Mikoyan, V. D., Chepurnov, S. A. and Chepurnova, N. E. (2001) Role of nitric oxide and lipid peroxidation in mechanisms of febrile convulsions in Wistar rat pups. Bull. Exp. Biol. Med., 131, 47-49,
Liu, L., Yang, T., Bruno, M. J., Andersen, O. S, and Simon, S. A. (2004) Voltage-Gated ion Channels in Nociceptors: Modulation by cGMP. J. Neurophysiol., 92, 2323-2332.
Mery, F., Belay, A. T., So, A. K., Sokolowski, M. B. and Kawecki, I. J, (2007), Natural polymorphism affecting learning and memory in Drosophila. Proc Natl Acad U S A 104, 13051-5.
Michelakis, E. D., McMurtry, M. S., Sonnenberg, B. and Archer, S. L. (2003). The NO− K+ channel axis in pulmonary arterial hypertension. Activation by experimental oral therapies, Adv Exp Med Biol 543, 293-322,
Michelakis, E. D., McMurtry, Wu, X. C., Dyck, J. R., Moudgil, R., Hopkins, T. A., Lopaschuk, G. D., Puttagunta, L., Waite, R. and Archer, S. L. (2002). Dichloroacetate, a metabolic modulator, prevents and reverses chronic hypoxic pulmonary hypertension in rats: role of increased expression and activity of voltage-gated potassium channels. Circulation 105, 244-50.
Milton, S. L. and Lutz, P. L. (2005), Adenosine and ATP-sensitive potassium channels modulate dopatnine release in the anoxic turtle (Tracheniys scripta) striatum. Am J Physiol Regul Integr Comp Physiol 289, R77-83.
Milton, S. L., Thompson, J. W. and Lutz, P. L. (2002). Mechanisms for maintaining extracellular glutamate levels in the anoxic turtle striatum, Am J Physiol Regul Integr Comp Physiol 282, R1317-23.
Nilson, T. L., Sinclair, B. J. and Roberts, S. P. (2006). The effects of carbon dioxide anesthesia and anoxia on rapid cold-hardening and chill coma recovery in Drosophila melanogaster. J Insect Physiol 52, 1027-33.
Ogawa, S., Kitao, Y. and Hori, O. (2007). Ischemia-induced neuronal cell death and stress response. Antioxid Redox Signal 9, 573-87.
Osborne, K. A. et al., (1997) Science. 277, 834.
Patel, A. I. and Diamond, J. (1997) Activation of Guanosine 3′,5′-Cyclic Monophosphate (cGMP)-Dependent Protein Kinase in Rabbit Aorta by Nitroglycerin and Sodium Nitroprusside, J Pharmacol. Exp. Ther., 283, 885-893.
Pereira, H. S. and Sokolowski, M. B. (1993). Mutations in the larval foraging gene affect adult locomotory behavior after feeding in Drosophila meglanogaster. Proc Natl Acad Sci U S A 90, 5044-6.
Perez-Pinzon, M. A., Rosenthal, M., Sick, T. J., Lutz, P. L., Pablo, J. and Mash, D. (1992), Downregulation of sodium channels during anoxia: a putative survival strategy of turtle brain. Am J Physiol 262, R712-5.
Ramirez, J. M., Elsen, F. P. and Robertson, R. M. (1999) Long-term effects of prior heat shock on neuronal potassium currents recorded in a novel insect ganglion slice preparation. J. Neurophysiol., 81, 795-802.
Raizen, D. M., Cullison, K. M., Pack, A. I, and Sundaram, M. V, (2006). A novel gain-of-function mutant of the cyclic GMP-dependent protein kinase egl-4 affects multiple physiological processes in Caenorhabditis elegans. Genetics 173, 177-87.
Reaurne, C. J. and Sokolowski, M. B. (2009). cGMP-dependent protein kinase as a moditier of behaviour, Handb Exp Pharmacol, 423-43.
Renger, J. J. et al., J Neurosci. 19, RC28 (1999).
Robertson, R. M. (2004) Thermal stress and neural function: adaptive mechanisms in insect model systems. Journal of Thermal Biology, 29, 351-358.
Ropero, A. B., Fuentes, E., Rovira, J. M., Ripoll, C., Soria, B. and Nadal, A. (1999). Non-genomic actions of 17beta-oestradiol in mouse pancreatic beta-cells are mediated by a cGMP-dependent protein kinase. J Physiol 521 Pt 2, 397-407.
Sachidhanandam, S. B., Low, K. S, and Moochhala, S. M. (2002) Naltrexone attenuates plasma nitric oxide release following acute heat stress. Eur. J. Pharmacol., 450, 163-167.
Sandberg, M., et al., FEBS Lett (1989) 251, 191-196.
Scheiner, R., Sokolowski, M. B. and Erber, J. (2004). Activity of cGMP-dependent protein kinase (PKG) affects sucrose responsiveness and habituation in Drosophila melanogaster, Learn Mem 11, 303-1.1,
Schiffmann, Desdouits, Menu, Greengard, Vincent, J., Vanderhaeghen, J. and Girault, J. (1998) Modulation of the voltage gated sodium current in rat striatal neurons by DARPP 32, an inhibitor of protein phosphatase. Eur. J. Neurosci., 10, 1312-1320.
Sick, T. J., Lutz, P. L, LaManna, J. C. and Rosenthal, M. (1982). Comparative brain oxygenation and mitochondrial redox activity in turtles and rats. J Appl Physiol 53, 1354-9.
Sokolowski, M. B. (1980). Foraging strategies of Drosophila melanogaster: a chromosomal analysis, Behav Genet 10, 291-302,
Sonijen, G. G. (2002) Ion regulation in the brain: implications for pathophysiology. Neuroscientist, 8, 254-267.
Somjen, G. G. (2001) Mechanisms of spreading depression and hypoxic spreading depression-like depolarization. Physiol. Rev., 81, 1065-1096.
Taylor, M. S., Okwuchukwuasanya, C., Nickl, C. K., Tegge, W., Brayden, J. E. and Dostmann, W. R. (2004) Inhibition of cGMP-dependent protein kinase by the cell-permeable peptide DT-2 reveals a novel mechanism of vasoregulation. Mol. Pharmacol., 65, 1111-1119.
Tryba, A. K. and Ramirez, J. M. Neurphysiol, 92, 2844 (2004).
Ultsch, G. Z. (2006). The ecology of overwintering among turtles: where turtles overwinter and its consequences. Biol Rev Camb Philos Soc 81, 339-67.
Wang, J., Zhou, Y., Wen, H. and Levitan, I. B. (1999) Simultaneous binding of two protein kinases to a calcium-dependent potassium channel, J. Neurosci., 19, RC4.
Wang, J. W., Humphreys, J. M., Phillips, J. P., Hilliker, A. J. and Wu, C. F. (2000) A novel leg-shaking Drosophila mutant defective in a voltage-gated K(+) current and hypersensitive to reactive oxygen species. J. Neurosci., 20, 5958-5964.
Weckstrom, M. and Laughlin, S. B. (1995), Visual ecology and voltage-gated ion channels in insect photoreceptors. Trends Neurosci 18, 17-21.
White, R. E. (1999) Cyclic GMP and ion channel regulation. Advances in Second Messenger and Phosphoprotein Research, 33, 251-277.
White, R. E., Shcherbatko, A. D., Lincoln, T. M., Schonbrunn, A. and Armstrong, D. L. (1993) Potassium channel stimulation by natriuretic peptides through cGMP-dependent dephosphorylation. Nature, 361, 263-266.
Wingrove, J. A. and O'Farrell, P. H. (1999), Nitric oxide contributes to behavioral, cellular, and developmental responses to low oxygen in Drosophila. Cell 98, 105-14.
Zeng, H., Fei, H. and Levitan, I. B. (2004) The slowpoke channel binding protein Slob from Drosophila melanogaster exhibits regulatable protein kinase activity. Neurosci. Lett., 365, 33-38.
Zhou, X. B., Schlossmann, J., Hofmann, F. and Korth, M. (1996) Protein phosphatase 2A is essential for the activation of Ca2+-activated K+ currents by cGMP-dependent protein kinase in tracheal smooth muscle and Chinese hamster ovary cells. J. Biol. Chem., 271, 19760-19767.
Zhou, X. B., Schlossmann, J., Hofmann, F., Ruth, P. and Korth, M. (1998) Regulation of stably expressed and native BK channels from human myometrium by cGMP- and cAMP-dependent protein kinase. Pflugers Arch., 436, 725-734.

What is claimed is:

1. A pharmaceutical composition comprising: a pharmacological activator of the PKG pathway in an amount effective for treating or preventing one or both of neural anoxia and reperfusion injury; and an excipient.

2. A pharmaceutical composition according to claim 1, wherein the pharmacological activator of the PKG pathway is a PKG activator.

3. A pharmaceutical composition according to claim 2, wherein the pharmacological activator is selected from the group consisting of:
8-bromo-PET-cyclic GMPS;
8-pCPT-cylic GMPS, TEA;
8-Br-cGMPS, Na.

4. A pharmaceutical composition according to claim 1, wherein the pharmacological activator is a cGMP-specific agonist.

5. A pharmaceutical composition according to claim 1, wherein the pharmacological activator activates K+ ion channel function.

6. A pharmaceutical composition according to claim 1, wherein the pharmacological activator is a protein phosphatase activator.

7. A pharmaceutical composition according to claim 6, wherein the protein phosphatase activator is selected from the group consisting of PTPA.

8. A pharmaceutical composition according to claim 1, wherein the pharmacological activator is a PDE inhibitor selected from the group consisting of sildenafil citrate (Viagra).

9. A pharmaceutical composition according to claim 1 comprising: a nucleic acid encoding a pharmacological activator of the PKG pathway in an amount effective for treating or preventing one or both of neural anoxia and reperfusion injury.

10. A method of treating or preventing a medical condition in a patient comprising administering to the patient a therapeutically effective amount of a pharmacological activator of the PKG pathway, wherein the medical condition is selected from the group consisting of damage from environmental hypoxia, spinal cord injury, and stroke.

11. The method of claim 10, wherein the pharmacological activator of the PKG pathway is selected from the group consisting of a PKG activator; a cGMP-specific agonist; an activator of K+ ion channel function; a protein phosphatase activator; and a sGC activator.

12. The method of claim 10, wherein the pharmacological activator is selected from the group consisting of:
8-bromo-PET-cyclic GMPS;
8-pCPT-cylic GMPS,TEA;
8-Br-cGMPS, Na;
PTPA;
Sildenafil Citrate (Viagra).

13. The method of claim 10, wherein the pharmacological activator of the PKG pathway is a nucleic acid encoding a pharmacological activator of the PKG pathway.

14. A method of providing one or both of neural anoxia and reperfusion injury protection in a patient comprising administering to the patient a therapeutically effective amount of a pharmacological activator of the PKG pathway.

15. The method according to claim 14 wherein the neural anoxia or reperfusion injury protection results from an increase in potassium ion channel conductances.

16. The method according to claim 14, wherein providing neural anoxia protection includes providing at least an effect selected from the group consisting of: lowering the level of oxygen at which neural function becomes abnormal; decreasing the amount of cell death from lack of oxygen and reperfusion.

17. A method of using the pharmaceutical composition of claim 1 treat or prevent a medical condition selected from the group consisting of damage from environmental hypoxia, spinal cord injury, and stroke.

18. A method of using the pharmaceutical activator of the PKG pathway of claim 2 to provide neural anoxia protection, reperfusion protection or a combination thereof.

19. The method of claim 18 wherein the neural anoxia protection, reperfusion injury protection or a combination thereof results from an increase in potassium ion channel conductances.

20. The method according to claim 18, wherein providing neural anoxia protection includes providing at least one effect selected from the group consisting of: lowering a level of oxygen at which neural function becomes abnormal; decreasing an amount of cell death from lack of oxygen and reperfusion or a combination thereof.
